(19) United States
(12) Patent Application Publication
(10) Pub. No.: US 2005/0025804 A1
(43) Pub. Date: Feb. 3, 2005

(54) Reduction of adverse inflammation

(76) Inventor: Adam Heller, Austin, TX (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/894,573

(22) Filed: Jul. 19, 2004

Related U.S. Application Data
(60) Provisional application No. 60/539,695, filed on Jan. 27, 2004.
(60) Provisional application No. 60/503,200, filed on Sep. 15, 2003.
(60) Provisional application No. 60/490,767, filed on Jul. 28, 2003.

(51) Int. Cl. A61K 38/44; A61K 31/555; A61F 2/00

(52) U.S. Cl. 424/423; 424/94.4; 514/185; 514/410

(57) ABSTRACT

Reduction of the likelihood of adverse inflammatory reaction to an implant or a transplant is achieved through several mechanisms including the catalysis of isomerization of peroxynitrite by a hydrogel-bound peroxynitrite isomerization catalysts. A second mechanism controls acceptable and unacceptable dimensions of surface features of implants, such as vascular stents. A third mechanism fabricates implants from materials which are substantially free from alloys transition metals which produce ions of which catalyze cell killing radical formation.

### CROSS-REFERENCES TO RELATED APPLICATIONS

This application claims the benefit of the following three U.S. Provisional Application Nos. 60/490,767 (Attorney Docket No. 021821-000200US), filed on Jul. 28, 2003; 60/503,200 (Attorney Docket No. 021821-000210US), filed on Sep. 15, 2003; and 60/539,695 (Attorney Docket No. 021821-000300US), filed on Jan. 27, 2004, the full disclosures of which are incorporated herein by reference. The disclosure of this application is also related to U.S. Patent Application No. 10/______ (Attorney Docket No. 021821-000230US), filed on the same day as the present application, the full disclosure of which is incorporated herein by reference.

### FIELD OF THE INVENTION

The present invention relates generally to implantable medical devices and methods for their fabrication and use. In particular, the present invention relates to apparatus, coatings, and methods for alleviating adverse inflammation which can occur upon implantation or transplantation of medical devices and transplantation structures.

Adverse inflammatory reaction to implants and transplants. Recognition of implants or transplants as foreign bodies by the immune system triggers the recruitment of killer cells to their host tissue interface. These cells release an arsenal of chemical weapons, killing cells of the host tissue and/or of the transplant. The killing is an amplified feedback loop involving process, as the killed cells release chemotactic molecules and debris, their release further increasing the number of the recruited cells.

Coronary stents, adverse inflammation and restenosis. Vascular stents are exemplary implants. Of these, coronary stents are implanted to alleviate insufficient blood supply to the heart. Some of the recipients of coronary stents develop in-stent restenosis, the narrowing of the lumen of the coronary artery at the site of the stent, typically through neointimal hyperplasia, a result of the proliferation of fibroblasts and smooth muscle cells. (See for example, V. Rajagopal and S. G. Rockson, “Coronary restenosis: a review of mechanism and management” The American Journal of Medicine, 2003, 115(7), 547-553)). The presence of macrophages and neutrophils at implants, including coronary stents, has been documented. (See, for example, F. G. Welt et al., “Leukocyte recruitment and expression of chemokines following different forms of vascular injury” Vasc. Med. 2003, 8(1), 1-7.) It has also been reported that hematopoietic cells of monocyte/macrophage lineage populate the neointima in the process of lesion formation. Furthermore, macrophages have been proposed to be precursors of neointimal myofibroblasts after thermal vascular injury (A. Bayes-Genis et al., “Macrophages, myofibroblasts and neointimal hyperplasia after coronary artery injury and repair” Atherosclerosis, 2002, 163(1), 89-98)). According to reported theories and models, such as those of J. Y. Jeremy et al, “Oxidative stress, nitric oxide, and vascular disease” J. Card. Surg. 2002, 17(4) 324-7; G. M. Jacobson et al., “Novel NAD(P)H oxidase inhibitor suppresses angioplasty-induced superoxide and neointimal hyperplasia of rat carotid artery” Circ. Res. 2003, 92(6), 637-43; T. Bleeke et al., “Catecholamine-induced vascular wall growth is dependent on generation of reactive oxygen species” Circ. Res. 2004, 94(1), 37-45)), by which this invention is not to be limited, the superoxide radical anion, O₂^{·-}, is among the key risk factors for cardiovascular disease. Cardiovascular diseases, where O₂^{·} is a risk factor, include restenosis following balloon angioplasty, atherogenesis, reperfusion injury, angina and vein graft failure.

Acceptable and unacceptable micro-roughness of medical implants. It is known that mechanically polished, electrochemically polished, or ion or electron beam or plasma polished surfaces of implants are less likely to cause adverse inflammatory reaction that surfaces that were not polished. This is the case, for example, of polished versus unpolished coronary stents, the likelihood of restenosis increasing steeply when unpolished stents are implanted. See, for example, Kirkpatrick et al., “Method and system for improving the effectiveness of medical stents by the application of gas cluster ion beam technology” U.S. Pat. No. 6,676,989. The dimension of the unacceptable or acceptable residual surface features of medical implants has, however, not been known or specified. Excessive polishing of stents is costly and unnecessary; inadequate polishing can increase the frequency of restenosis. Polishing to avoid even the smallest detectable surface features is costly. Hence, there is a need to specify the acceptable micro-roughness.

Catalysis of Conversion of Peroxynitrite to Nitrate and its Beneficial Effect. The cell-killing oxidizer's precursor, the peroxynitrite anion, ONOO⁻, is a prime weapon of killer cells, particularly monocyte derived macrophages and macrophage-derived cells, such as giant cells, known to infuse and kill cells of the transplant. Because the peroxynitrite anion is much less reactive than the ^{·}OH radical, and is also less reactive than the CO₃^{·-} radical, its half-life in plasma, the fluid between the cells in living tissues, is much longer. It lives long enough for the diffusion distance in plasma to equal or exceed the distance between the killer cells, located in or near the chemotactic front and the still living cells. This front is initially at or near the macrophage-exposed surface of the transplant, but as cells are killed, it propagates, with its macrophages and other killer cells, deeper into the transplanted tissue or organ. Therefore, the cell killing macrophages infuse the transplant, accumulating, fusing and/or spreading in the acute transplant-rejection phase. According to D. Jourd'heuil et al. Journal of Biological Chemistry, 2001, 276, 28799-28805 the peroxynitrite anion is a potent cell killer because it can diffuse into the cell, where it decomposes to form an ^{·}OH radical and nitrogen dioxide, ^{·}NO₂.

This would indeed be the case in the absence of bicarbonate anions. In their presence, ^{·}OH, if generated, reacts according to Reaction 5 to form CO₃^{·-}, which is less reactive, but has a half life of ˜1 ms and L of a few μm, long enough to reach oxidizable components of cells, making it highly toxic.
^{·}OH+HCO₃⁻→CO₃^{·-}+H₂O (5)
The application of peroxynitrite to nitrate conversion catalysts in preventing adverse implant or transplant associated inflammation has not been reported, even though the beneficial anti-inflammatory effect of porphyrin-based catalysts of peroxynitrite to nitrate isomerization has been described. Thus, alleviation of inflammatory transplant rejection by isomerization of peroxynitrite anions to nitrate anions by systemically, preferably parenterally, administered iron porphyrins has been disclosed. It has also been disclosed that the killing of cells can be stopped by decomposing, by preventing the generation of, or by scavenging, the nitric oxide precursor radical; or by preventing the generation of, or by scavenging, the superoxide radical anion. Of these, the second option, preventing the generation of, or scavenging nitric oxide has generally been unsuccessful, because nitric oxide has essential biological functions, such as vasodilation.

Riley et al. WO1998/43637 disclosed therapeutic peroxynitrite decomposition catalysts. Their compounds were transition metal containing macrocycles, among which an iron porphyrin was uniquely effective. Stern & Salvemini U.S. Pat. No. 6,245,758 applied peroxynitrite decomposition catalysts in pharmaceutical compositions. The catalysts were transition metal complexes, such as those of porphyrins and phthalocyanines, the fastest being macrocyclic complexes of iron. Ruthenium phthalocyanines were also disclosed. One of their most effective, fastest catalysts was acetato (5,10,15,20-tetrakis(N-methyl-4-pyridyl)porphinato) iron (III) tetratosylate, termed Fe(III)TMPyP, (rate constant 2.75×10⁶ M⁻¹ sec⁻¹); another was acetato-5,10,15,20-tetrakis(3,5-disulfonatomesityl) porphyrin iron (III) octasodium salt, termed (Fe(III)TMPS), (rate constant 2.06×10⁶ M⁻¹ sec⁻¹). In general, the therapeutic catalysts were water soluble, not immobilized. Treatable conditions according to Riley et al. WO1998/43637 included myocardial ischemia, inflammation, ischemic reperfusion and others. The cytotoxic effects of stimulated neutrophils or peroxynitrite on endothelial cells was determined using a ⁵¹Cr-release assay as described by Moldow et al. (Meth. Enzymol. 105, 378-385, [1984]). FIG. 5 of Riley shows peroxynitrite-mediated endothelial cell injury in a cell culture; FIG. 7 shows inhibition of neutrophil-mediated injury to human aortic endothelial cells by Fe(TMPyP), their fastest catalysts. Other cells were also protected against peroxynitrite anions. The inventors cite Beckman et al. “Apparent hydroxyl radical production by peroxynitrite: Implication to endothelial injury from nitric oxide and superoxide” PNAS 87, 1620-1624, 1990, pointing out that the ONOO⁻ anion is more damaging to cells than the ^{·}OH radical itself, because of its longer life, longer diffusion length and its ability to pass cell membranes. Effectiveness in vivo was shown by prevention of carrageenan-induced paw edema in rats and prevention of intestinal damage by endotoxin in rats. Only the fast catalytic iron porphyrins were effective; their non-catalytic zinc counterparts were not. U.S. Pat. No. 6,448,239 and US Pat. Appl. 20030055032 of Groves & Moeller also describes water-soluble macrocyclic complexes of transition metals that are peroxynitrite decomposition catalysts and their use as drugs, usually orally administered. They include porphyrins and phthalocyanins. The preferred ones are solubilized in water by attached PEG functions. They are said to be useful for treating any of a very large number of afflictions, diseases and disorders. Administration to patients undergoing any of a very large number of surgical procedures, including transplantation, is also mentioned.

T. P. Misko et al. state in their article “Characterization of the cytoprotective action of peroxynitrite decomposition catalysts” Journal of Biological Chemistry, 1998, 273, 15646-15653 that “The formation of the powerful oxidant peroxynitrite (PN) from the reaction of superoxide anion with nitric oxide has been shown to be a kinetically favored reaction contributing to cellular injury and death at sites of tissue inflammation. The peroxynitrite molecule is highly reactive causing lipid peroxidation as well as nitration of both free and protein-bound tyrosine. We present evidence for the pharmacological manipulation of peroxynitrite with decomposition catalysts capable of converting it to nitrate. In target cells challenged with exogenously added synthetic peroxynitrite, a series of metalloporphyrin catalysts (5,10,15,20-tetrakis(2,4,6-trimethyl-3,3-disulfonatophenyl)-porphyrinato iron(III) (FeTMPS); 5,10,15,20-tetrakis(4-sulfonatophenyl)porphyrinato iron(III) (FeTPPS); 5,10,15,20-tetrakis(N-methyl-4′-pyridyl)porphyrinato iron(III) (FeTMPyP)) provided protection against peroxynitrite-mediated injury with EC50 values for each compound 30-50-fold below the final concentration of peroxynitrite added . . . ” “Our studies provide compelling evidence for the involvement of peroxynitrite in cytokine-mediated cellular injury and suggest the therapeutic potential of peroxynitrite decomposition catalysts in reducing cellular damage at sites at sites of inflammation.” Jeremy et al., wrote that “ . . . ·O₂⁻ reacts with nitric oxide (NO) to form peroxynitrite (ONOO⁻) resulting in a depletion of endogenous vascular NO, which is now firmly associated with CVD (cardiovascular disease). Furthermore, risk factors for CVD, in particular diabetes mellitus, dyslipidemia, and hyperhomocysteinemia are all associated with oxidative stress. (Jeremy J. Y. et al. “Oxidative stress, nitric oxide, and vascular disease” J. Card. Surg. 2002, 17, 324-7).

Peroxynitrite scavenging drugs. Bridger et al U.S. Pat. Appl. 20020193363 disclose that administration of ^{·}NO scavengers can reduce the inflammatory damage in coronary bypass artery grafting (CABG) associated with macrophages and with other agents, for example through its “breaking down” to the toxic peroxynitrite anion OONO⁻, mistakenly termed a “radical”. To modulate the inflammation they administer to the patient [Ruₐ(X_{b}L)_{c}Y_{d}Zₑ]ⁿ where X is a cation L and Y are ligands, Z is a halide or pseudohalide. They consider their medicine to be useful in treating a very large number of diseases. They point out that systemic inhibition of iNOS (induced nitric oxide synthase) by drugs has an adverse effect, because ^{·}NO has important physiological functions. They prefer, instead, to scavenge NO. Administration of their drug is usually parenteral (tablet, capsule, suppository etc.) A specific experimental ^{·}NO scavenging Ru compound was AMD6621, [Ru(H₃dtpa)Cl] dtpa=diethylenetriamine-pentaacetic acid. It was administered to dogs undergoing cardiopulmonary bypass surgery.

Nitric oxide scavenging drugs. To lower the level of ^{·}NO, Lai & Wang U.S. Pat. Application 20030087840 scavenge it with dithiocarbamates, primarily those of iron, but also including those of ruthenium and of other metals. Usually the ^{·}NO-scavengers are bound to or are co-administered with non-steroid anti-inflammatory drugs (NSAID) like Naproxen, reducing their damage to the digestive tract. In U.S. Pat. Applications 20030087840 and 20030040511 Lai reduced free radical levels in mammals using a free radical scavenger, particularly the iron dithiocarbamate complex, transported in the bloodstream. Ruthenium complexes are also disclosed. Administration is oral, enteral or parenteral (tablets, capsules, syrups, suppositories etc.). Lai U.S. Pat. No. 6,469,057 reduced radical levels, including ^{·}NO levels in mammals by administering an iron dithiocarbamate complex. Graft vs. host disease, transplant rejection are among the many diseases treated. Lai & Wang U.S. Pat. No. 6,407,135 use conjugates of nitric oxide scavengers and NSAID as in 20030087840. Lai U.S. Pat. No. 6,316,502 discloses a dithiocarbamate disulfide dimer co-administered with an agent inhibiting expression of nitric oxide synthases, such as in macrophages and such as associated with transplant rejection. Lai & Vassilev U.S. Pat. No. 6,093,743 disclosed dithiocarbamate disulfide drugs comprising co-administered with agents inhibiting the activation of nitric oxide synthases. Lai U.S. Pat. No. 5,916,910 discloses conjugates of nitric oxide scavengers, particularly dithiocarbamates, and NSAIDs lowering the side effects of NSAIDs. Lai & Vassilev U.S. Pat. No. 6,589,991 disclose as above, dithiocarbamate disulfide dimers that not only reduce ^{·}NO levels by scavenging, but also scavenge free iron ions. They inhibit nuclear factor kappa B pathways. Lai & Vassilev U.S. Pat. No. 6,596,770 co-administered a dithiocarbamate disulfide with a drug capable of inactivating species inducing nitric oxide synthase.

The implantation of some elemental metals and of alloys, such as copper and its alloys, causes adverse inflammation. Adverse inflammation for implants, such as stents, made of stainless steels, cobalt-chromium, and nickel-titanium, is less frequent than for copper, but it does occur, and when stents are implanted it frequently leads to restenosis. Zirconium alloys and ceramic zirconia, ZrO₂, are used in orthopedic implants and in coatings of orthopedic implants. Their application in stents has been suggested by Davidson, U.S. Pat. Nos. 5,169,597, 5,496,359, 5,588,443, 5,647,858 and 5,649,951 and by Hunter et al., U.S. Pat. Nos. 6,447,550 and 6,585,772.

U.S. Pat. Nos. 5,649,951; 5,647,858; 5,588,443; and 5,496,359 describe stents and/or stent coatings composed of an alloy of hafnium containing zirconium. No disclosure of reducing transition metals in surface oxides and nitrides is provided.

### BRIEF SUMMARY OF THE INVENTION

The present invention provides medical implants comprising, composed of, or coated by materials which inhibit significant adverse inflammation of tissue around the implant. In particular, the present invention employs materials and methods which reduce the likelihood of adverse inflammation. Adverse inflammation can result, for example, in the killing of cells of healthy tissue of a transplant, of host tissue near a transplant, or of host tissue near an implant. It can also result, through the consumption or generation of chemicals by inflammatory cells, in an unwanted change of the concentration of an analyte measured by an implanted sensor or monitor. Furthermore, inflammation can result in reduction of the flux of nutrients and/or O₂ to cells or tissue or organ in implanted sacks, protecting the cells in the sack from the chemical arsenal of killer cells of the immune system. The cells, or tissue or organ in the sack replace a lost or damaged function of the human body. Adherent inflammatory cells, or fibrotic or scar cells, growing on the sack after adverse inflammatory reaction, can starve the cells in the sack.

Adverse inflammation, often associated with an inflammatory flare-up in which a large number of healthy cells of normal tissue are killed, is avoided or reduced by avoidance of the initiation, or the disruption, of the feedback loop, elements of which include the release of pre-precursors of cell killing radicals by inflammatory killer cells, such as macrophages or neutrophils; release of chemotactic molecules and/or debris by the killed cells; and the recruitment of more killer cells, releasing more of the pre-precursors of the cell killing radicals.

Medical and cosmetic implants, termed here “implants”, are widely used, and novel implants are being introduced each year. Examples of the implants include vascular implants; auditory and cochlear implants; orthopedic implants; bone plates and screws; joint prostheses; breast implants; artificial larynx implants; maxillofacial prostheses; dental implants; pacemakers; cardiac defibrillators; penile implants; drug pumps; drug delivery devices; sensors and monitors; neurostimulators; incontinence alleviating devices, such as artificial urinary sphincters; intraocular lenses; and water, electrolyte, glucose and oxygen transporting sacks in which cells or tissues grow, the cells or tissues replacing a lost or damaged function of the human body.

In the first of its several aspects, this invention provides materials and methods for avoidance or reduction of adverse inflammatory response in which healthy cells near the implant or in some cases transplant structures are killed. In its second aspect, it provides materials and methods for avoidance or reduction of the inaccuracy the measurement of the concentration of a chemical or biochemical, or a physiological parameter such as temperature, flow or pressure, by an implanted sensor or monitor, associated with an inflammatory response, where the local consumption or the local generation of a chemical or biochemical is changed by recruited inflammatory cells, or where these cells locally change a physiological parameter. In its third aspect, this invention provides materials and methods for the maintenance of a flux of nutrient chemicals, oxygen and other essential chemicals and biochemicals into implanted sacks, containing living cells or tissue, the function of which is to substitute for lost or damaged tissue, organs or cells of an animal's body, particularly the human body. If the implanted sack would cause and inflammatory response, in which normal neighboring cells would be killed, then the proliferation cells produced in the repair of the lesion would consume chemicals and reduce the influx of chemicals, such as nutrients or oxygen.

Examples of organs and other transplant structures that are transplanted include the kidney, the pancreas, the liver, the lung, the heart, arteries and veins, heart valves, the skin, the cornea, various bones, and the bone marrow. Adverse inflammatory reaction to a transplant can cause not only the failure of the transplanted organ, but can endanger the life of the recipient.

The carbonate radical anion, CO₃^{·-} is the most potent cell killing species generated of the intermediates released by the killer cells. The hydroxyl radical, ^{·}OH, is another potent cell killer. CO₃^{·-} and ^{·}OH are generated by reactions of a common precursor, the peroxynitrite anion, ONOO⁻. This anion is formed when the superoxide radical anion, ·O₂⁻, combines with nitric oxide, ^{·}NO.

Thus, in a first aspect, the present invention prevents or inhibits adverse inflammation, in which healthy cells of normal tissue would otherwise be killed, by accelerating the isomerization of ONOO⁻ to NO₃⁻ using an immobilized catalyst. The isomerization catalyst is immobilized on or over at least a portion of the implant, typically being incorporated in a hydrogel coated or otherwise immobilized or localized over at least a portion of the surface of the implant or transplant. The hydrogel is permeable to ONOO⁻ and/or to NO₃⁻.

The implant or transplant is thus fabricated or modified to promote the isomerization of peroxynitrite anion to nitrate anion. At least a portion of a surface of the implant or transplant is coated with a catalyst which promotes said isomerization, where the catalyst is usually a protein, such as an enzyme, and/or other metal-containing complex. Preferred catalyst compositions comprise a permeable hydrogel containing a porphyrin and/or phthalocyanins, such as iron, manganese, or the like.

Methods for inhibiting inflammation associated with implantation or transplantation in a patient therefore comprise coating at least a portion of an implant device or transplantation structure, such as any of the organs listed in the present application, with a material which catalyzes the isomerization of peroxynitrite anion to nitrate anion. Preferred exemplary compositions for providing such catalyst coating are described above.

The present invention still further comprises hydrogels for coating a medical implant or transplant which promotes the isomerization of peroxynitrite anion to nitrate anion. Exemplary and preferred hydrogels are described above.

In a second aspect, the present invention provides for prevention or alleviation of adverse inflammatory reaction to an implant, leading in the exemplary case of coronary stents to restenosis, by dimensional control of features protruding from the surface of the implant. Surface features having dimensions similar to those of common human pathogenic bacteria are avoided. Features much larger or much smaller are, however, acceptable. The present invention thus provides both the medical implants and methods for fabricating such implants to control the density of surface features as noted above. Surface features in the range from 0.1 μm to 100 μm will be limited to threshold surface densities below 1000 features per mm². Preferred and exemplary size ranges and further surface densities are set forth in detail below.

In a third aspect, the present invention provides for the manufacture, fabrication, and/or modification of medically implantable devices in order to promote prevention, alleviation, and/or reduction of the likelihood of adverse inflammation of tissue surrounding an implant. Medical implants will be provided having surface areas which are substantially free from transition metals which form dissolved ions which catalyze the formation of cell-killing radicals, as described in more detail below. Exemplary transition metals which lead to such catalyzes include cooper, iron, cobalt, nickel, and other materials of the type which are commonly found in implantable medical devices, such as vascular and other stents. According to the present invention, such transition metals will be present at or near the surface of the medical implant at an atomic percent below 1 percent, preferably below 0.1 atomic percent. Preferably, the medical implants may be formed from other transition metals which do not promote such catalysts, including yttrium, zirconium, hafnium, magnesium, calcium, aluminum, lithium, scandium., and alloys and/or oxides thereof. Preferred implants will be composed of a metal or metal alloy having a 20% or greater elongation failure at room temperature. An exemplary medical implant comprises a stent or other implantable device composed of at least 95 atomic percent zirconium and from 0 to 5 percent hafnium.

The present invention further comprises methods for forming such medical implants composed of alloys which do not catalyze the formation of cell-killing radicals. The implants and methods of the present invention preferably employ alloys with mechanical properties appropriate for their drawing to fine wires, such as about 0.25 mm diameter wires, not containing, or containing less than 3 atom % of a transition metal, the ions of which can be electroreduced or electrooxidized in an aqueous pH 7.2-7.4, 0.14 M NaCl containing buffer solution at 37° C. An example of such an alloy is that of zirconium and hafnium, preferably of the composition ZrₖHfₘ where k is between about 94 atom % and about 100 atom %, m is between about 0 atom % and about 6 atom %. Unlike the stainless steels, cobalt-chromium alloys and nickel titanium alloys of which many metallic implants, including vascular stents, are made, neither the oxides of the oxidized surfaces of the inventive alloys, nor their dissolution products in physiological solution catalyze redox reactions, such as those of H₂O₂ or ONOO⁻.

Examples of adverse inflammation treated or avoided through use or application of the materials and methods disclosed are inflammatory reaction to an implant, exemplified by restenosis near a cardiovascular stent; inflammatory rejection of transplanted tissue, organ, or cell; inflammation of a tissue or organ not infected by a pathogen, for example in immune, autoimmune or arthritic disease; inflammation following trauma, such as mechanical trauma, burn caused by a chemical, or by excessive heat, or by UV light, or by ionizing radiation; or persisting inflammation of the skin, mouth, throat, rectum, a reproductive organ, ear, nose, or eye following infection by a pathogen, after the population of the pathogen has declined to or below its level in healthy tissue.

### BRIEF DESCRIPTION OF THE DRAWINGS

FIG. 1 illustrates an exemplary medical implant fabricated in accordance with the principles of the present invention.

FIG. 2 is a detailed, cross-sectional view of a portion of the implant of FIG. 1, taken along line 2-2.

### DETAILED DESCRIPTION OF THE INVENTION

Terms and Definitions. Adverse inflammation or adverse inflammatory reaction is an inflammation other than inflammation to fight pathogens or mutated cells. Often large numbers of normal cells die in adverse inflammation.

Implant means a component, comprising man-made material, implanted in the body. The man made material can be a thermoplastic, a thermosetting or an elastomeric polymer; a ceramic; a metal; or a composite containing two or more of these.

Transplant means a transplanted tissue, a transplanted organ or a transplanted cell. The transplant can be an allograft or a xenograft. An allograft is a tissue or an organ transplanted from one animal into another, where the donor and the recipient are members of the same species. A xenograft is a tissue or an organ transplanted from one animal into another, where the donor and the recipient are members of different species. The animals are usually mammals, most importantly humans.

Chemotaxis is the migration of killer cells to the source of chemicals and/or debris from damaged or dead cells, usually damaged or killed by killer cells.

Killer cells are either cells generating chemicals or biochemicals that kill cells, or progenitors of the actual killer cells. The killer cells are usually white blood cells or cells formed of white blood cells. Macrophages, giant cells and cells formed of macrophages, as well as neutrophils, are examples of killer cells. The macrophages are said to be formed of monocytes in the blood.

Chemotactic recruitment means causing the preferred migration of killer cells, or progenitors of killer cells, to the implant or to the transplant and their localization in or near it. Chemicals and/or debris from killed cells of the tissue hosting the implant or the transplant, or from killed cells of the transplanted tissue or organ is chemotactic, meaning that the released molecules and/or debris recruits more killer cells or progenitors of killer cells.

Programmed cell death is normal orchestrated cell death in which the dead cell's components are so lysed or otherwise decomposed that few or no chemotactic molecules and/or debris are released.

Immobilized catalyst and insoluble catalyst mean a catalyst that is insoluble, or that dissolves, or that is leached, very slowly. A very slowly dissolving or leached catalyst is a catalyst less than half of which dissolves in one day, or is otherwise leached in one day, by a pH 7.2, 0.14 M NaCl, 20 mM phosphate buffer solution at 37° C. in equilibrium with air.

Plasma means the fluid bathing the implant or the transplanted tissue, organ or cell, and/or the intercellular fluid bathing the cells of the transplanted tissue, organ or cells.

Near the implant or near the transplant means the part of the tissue or organ hosting the implant or the transplant, located within less than 5 cm from the implant or the transplant, preferably within less than 2 cm from the implant or the transplant and most preferably within less than 1 cm from the implant or the transplant.

Permeable means a film or membrane in which the product of the solubility and the diffusion coefficient of the permeating species is greater than 10⁻¹¹ mol cm⁻¹s⁻¹ and is preferably greater than 10⁻¹⁰ mol cm⁻¹s⁻¹ and is most preferably greater than 10⁻⁹ mol cm⁻¹s⁻¹.

Hydrogel means a water swollen matrix of a polymer, which does not dissolve in an about pH 7.2-7.4 aqueous solution of about 0.14 M NaCl at about 37° C. in about 3 days. It contains at least 20 weight % water, preferably contains at least 40 weight % water and most preferably contains at least 60 weight % water. The polymer is usually crosslinked.

Recognition and the recruitment of inflammatory killer cells. Inflammation is generally associated with the recruitment of white blood cells, exemplified by leucocytes, such as neutrophils and/or monocytes and/or macrophages. The white blood cells secrete pre-precursors of potently cell killing oxidants. According to theoretical models, by which this invention is not to be limited, the rejection of transplants involves recognition, usually by lymphocytes, resulting, after multiple steps, in the killing of some cells of the transplant, then in the eventual chemotactic recruitment of killer cells by debris of the killed cells, and the killing of more cells by oxidants generated by the killer cells. The sequence of recruitment of killer cells, the killing of cells by the oxidants they secrete, the killing of more cells, the release of chemotactic chemicals and/or debris and the recruitment of an even greater number of killer cells constitutes an amplified feedback loop.

The arsenal of killer cells. The cell killing arsenal of the inflammatory cells, such as macrophages and neutrophils, consists of two radicals, the superoxide radical anion, ·O₂⁻ and nitric oxide, ^{·}NO. Superoxide radical anion is produced in the NADPH-oxidase catalyzed reaction of O₂ with NADPH. Nitric oxide is produced by the nitric oxide synthase (NOS) catalyzed reaction of arginine. The NOS of inflammatory cells is iNOS, inducible nitric oxide synthase. In the absence of scavenging reactants or enzymes accelerating their reactions these, they are relatively long lived, their half live equaling or exceeding a second. For this reason, their diffusion length, L, which is the square root of the product of their half life, τ_{1/2}, and their diffusion coefficient, D, which is about 10⁻⁵ cm² sec⁻¹ can also be long, equaling or exceeding 30 μm, a distance greater than the distance between the centers of large cells. Thus the pre-precursors secreted by nearby killer cells can reach and enter nearby tissue cells. The oxidant precursors, formed of the pre-precursors, include the peroxynitrite anion, ONOO⁻, and hydrogen peroxide, H₂O₂. These are also long-lived. At the physiological pH of 7.2-7.4, and in absence of enzymes accelerating their reaction, such as catalase or peroxidase in the case of H₂O₂, their τ_{1/2}/≧1 second, and their L≧30 μm. The ONOO⁻ precursor reacts with bicarbonate, HCO₃⁻, which abounds in tissues and cells, to form the potently oxidizing carbonate radical anion, CO₃^{·-} and nitrite anion, NO₂⁻. H₂O₂ may react with reductants to form hydroxide anion, OH⁻, and the hydroxyl radical, ^{·}OH, which reacts rapidly with HCO₃⁻ to form CO₃^{·-} and water. The τ_{1/2} of CO₃^{·--} is about 1 millisecond, and its L is about 1 μm. Thus, after a precursor enters a cell and reacts to form CO₃^{·-}, the CO₃^{·-} lives long enough to diffuse across distances approaching or equaling the dimension of the cell, allowing it to oxidize any of its oxidizable components. This makes it the premier killer of cells.

Potently cell killing CO₃^{·-} generated from its ONOO⁻ precursor and the importance of superoxide dismutase and/or superoxide dismutase mimics in reducing the killing of cells by CO₃^{·-}. The nature of the chemicals secreted by white blood cells, termed here pre-precursors, and the chemicals formed of these pre-precursors, termed here precursors, as well as the potently cell killing chemicals formed of the precursors, is known. The white blood cells generate two important pre-precursors the superoxide anion radical, O₂^{·-} and nitric oxide, ·NO. O₂^{·-} is believed to be generated by NADPH oxidase-catalyzed reduction of molecular oxygen, O₂ through exemplary Reactions 1 and 2. ^{·}NO is believed to be generated through nitric oxide synthase, NOS, catalyzed oxidation of arginine. The NOS of white blood cells is believed to be inducible nitric oxide synthase, iNOS.

The peroxynitrite anion, ONOO⁻, is a precursor of the potently cell killing CO₃^{·-} radicals. It is formed of O₂^{·-} and ^{·}NO through Reaction 1.
O₂⁻+^{·}NO→ONOO⁻ (1)

According to accepted models, cell killing CO₃^{·-} is generated from ONOO⁻ mostly through Reactions 2 and/or 3.
ONOO⁻+HCO₃⁻+H⁺→CO₃^{·-}+^{·}NO₂+H₂O (2)
ONOO⁻+2HCO₃⁻→2CO₃^{·-}+^{·}NO₂⁻+H₂O (3)

It has been proposed that ONOO⁻ decomposes in part to the hydroxyl radical, ^{·}OH, and to nitrogen dioxide, ^{·}NO₂. It has also been proposed that cells are killed mostly by ^{·}OH. The ^{·}OH radical reacts, however, promptly with the abundant, usually >10 mM, bicarbonate present in the cytoplasm of cells and in plasma, to form the highly toxic, longer lived, CO₃^{·-}, Thus, according to the best available models, by which this invention is not to be limited, irrespective of whether or not ^{·}OH is an intermediate, the cell killing species formed is CO₃^{·-}. The amount of O₂^{·-} available for generating ONOO⁻ is reduced when the O₂^{·-} is dismutated to H₂O₂ through a superoxide dismutase, SOD, catalyzed Reaction 4. Such dismutation reduces the availability of O₂^{·-} for the production of ONOO⁻, and thereby the killing of cells by its product, CO₃^{·-}
2·O₂⁻+2H⁺→H₂O₂+O₂ (4)

O₂^{·-}, ONOO⁻ and adverse inflammation. Adverse inflammatory response to chronic implants or transplants, leading, for example, to restenosis at sites of cardiovascular stents is associated with downstream products of reactions of the superoxide radical anion, particularly of ONOO⁻ and/or H₂O₂ formed by the dismutation of O₂^{·-}. The catalytic destruction of the O₂^{·-} and/or ONOO⁻ anions could alleviate or prevent undesired inflammation, inflammatory response to implants exemplified by restenosis, and/or acute inflammatory rejection of transplanted tissue or organs.

Proposed etiology of restenosis. Restenosis, such as in-stent proliferation of fibroblast and smooth muscle cells, is presently believed by the inventor herein to involve an inflammatory process, resulting in the killing of healthy cells of the coronary artery. The killing of the cells results in a lesion, which is repaired not by growth of normal endothelial cells, but by proliferating fibroblasts and smooth muscle cells, the cells causing the narrowing of the lumen of the artery in neointimal hyperplasia. The neointimal hyperplasia causing process may start, for example, with the recruitment of a few phagocytes, such as macrophages and neutrophils, by corroding microdomains, usually microanodes, of the transition metal comprising stent alloy, or by residual protruding features of the stent, particularly by features having dimensions and shapes resembling bacteria. Next, some of the chemical zones and/or protruding topographic features of the surface of the stent are covered by recruited phagosomes. In these, potent cell killing species, particularly CO₃^{·-} radicals, are generated from their macrophage and/or neutrophil generated ONOO⁻ precursor, eventually killing the phagosome. Killing results in the release of chemotactic molecules and/or debris, which attracts more macrophages and/or neutrophils. As a result, the surface of the stent becomes densely populated by these cells. For individual killer cells, the concentrations of ·O₂⁻ and ^{·}NO, the secreted pre-precursors of cell killing radicals, declines with the cube of the distance from the cell. Hence, individual macrophages or neutrophils are ineffective killers of cells other than the cells which are phagocytize. In contrast, when a surface is densely populated by macrophages or leucocytes, their concentration declines linearly with the distance from the macrophage or leucocyte covered surface. Hence, the radicals combine to form, with higher yield, ONOO⁻, the precursor of the highly toxic, cell killing, CO₃^{·-} and/or the potently oxidizing, possibly also formed, ^{·}OH. The killing of a massive number of the cells by the CO₃^{·-} and or ^{·}OH results in a lesion. The imperfect repair of the lesion by proliferating fibroblasts and smooth muscle cells results in restenosis, the narrowing of the lumen of the artery.

Adverse inflammation near implants. Inflammatory killer cells, like macrophages and neutrophils, evolved to destroy organisms recognized as foreign. They persistently try to destroy implants and can cause restenosis in stented blood vessels. They adhere to and merge even on implants said to be biocompatible, often forming large macrophage covered areas. Their presence on chronic implants usually leads to a permanent, clinically acceptable low level of inflammation, though in part of the orthopedic and other implants periodic adverse inflammatory flare-ups do occur.

The peroxynitrite anion precursor of the cell killing CO₃^{·-} and/or OH is produced in the combination of two macrophage-produced radicals, nitric oxide and superoxide radical anion (^{·}NO+O₂^{·-}→ONOO⁻). Nitric oxide is a short lived, biological signal transmitter. By itself it is not a strong oxidizer. ·O²⁻ is also not a potent oxidizer, behaving in some reactions as a reducing electron donor. The half lives of ^{·}NO and O₂^{·-} can be long, >1 second. The product of their combination, ONOO⁻, oxidizes, for example in Reactions 2 and/or 3, directly or through intermediate ^{·}OH, the bicarbonate anion HCO₃⁻, which is abundant in plasma, forming the highly toxic CO₃^{·-} radical.

When cells die naturally, by the orchestrated process of apoptosis, their decomposition products are not chemo-attractants of macrophages. In contrast, when cells are killed by the products of peroxynitrite, the chemicals and/or debris released are chemotactic for (chemically attract, or “recruit” more) macrophages. As a result a feedback loop, a flare up in which many cells are killed, can result. The killing of many cells can produce a lesion. As the killing of more cells leads to more debris and to the recruitment of even more macrophages, and as more macrophages are recruited, the damage is amplified and the size of the lesion is increased. The body's subsequent repair of the lesion can lead to the proliferation of cells and can underlie stent-caused restenosis. This self propagating, increasingly destructive process can be avoided by using the described materials, and disrupted, slowed, alleviated, or stopped by the disclosed ·O₂ dismutation and/or ONOO⁻ isomerization catalysts.

The catalyst can be coated on implants prior to their implantation, incorporated in the coating of the implant, or incorporated in the tissue proximal to the implant. Two groups of catalysts are particularly useful. The first, for ·O₂ dismutation, contains osmium, as described in co-pending U.S. Application No. 10/______ (Attorney Docket No. 021821-000230US), the full disclosure of which has been incorporated herein by reference. The second, for ONOO⁻ isomerization, are immobilized ONOO⁻ and/or NO₃⁻ permeable hydrogels, containing porphyrins and phthalocyanines of transition metals, particularly of iron and manganese, known to catalyze the peroxynitrite to nitrate isomerization.

Adverse inflammation in the acute rejection of transplants. As described above, white blood cells can kill cells of transplants. Their presence on transplants can cause a permanent, low-level inflammation, which can be tolerated and is clinically acceptable. In part of the transplants, it causes, however, inflammatory flare up and necrosis. The amplified cycle underlying the flare up and/or necrosis usually involves the generation of, and the killing of cells by, strong oxidants exemplified by products of reactions of the peroxynitrite anion, particularly CO₃^{·-} and/or ^{·}OH.

Immobile hydrogels catalyzing the isomerization of ONOO⁻ to NO₃⁻. Though it has been recognized that catalysis of processes reducing the concentration of the peroxynitrite anion or of its precursors by systemically administered water soluble catalyst molecules could be beneficial in treating a variety of inflammatory diseases, including the rejection of transplants, the use of hydrogels in which an immobilized catalyst accelerates the isomerization of ONOO⁻ to NO₃⁻ and in which are permeable to ONOO⁻ and/or to NO₃⁻ has not been proposed. Such a hydrogel can be applied on the implant or on or near the transplant.

According to this invention, the concentration of the peroxynitrite (OONO⁻) anions or of their precursors at, in, or near the transplant is lowered by a catalyst immobilized in, on or near the transplant. It has not been earlier recognized that cell death by inflammatory reaction to transplants could be reduced, alleviated or avoided by OONO⁻ concentration-reducing catalysts immobilized on, in, or near transplants. Also according to this invention, the immobilized catalyst is insoluble. The immobilized and insoluble catalyst reduces the concentration of the peroxynitrite anion mostly in, on, or near the transplant. There are significant advantages in using immobilized catalysts instead of the previously disclosed, systemically administered, soluble catalysts. For example, because the doses are lower when the catalyst is restricted to the site where it is needed, adverse side effects and systemic effects, caused by the higher doses of the systemically administered catalysts, are avoided. Furthermore, while the systemically administered catalysts were generally water soluble molecules, dispersions comprising small particles of metal oxides or metals can be used to reduce the concentrations of peroxynitrite anions or of is precursors on, in, or near transplants.

Catalysts coated on and/or slowly released from coatings on implants or transplants. Hydrogel-bound catalysts of the isomerization of OONO⁻ to NO₃⁻ are disclosed. The catalysts are intended to prevent, reduce or alleviate adverse inflammation near implants, or the inflammatory rejection of transplants. Preferably, the catalysts are immobilized in, on, or near the implant, or the transplanted tissue, organ, or cell.

These catalysts accelerate a reaction wherein the OONO⁻ precursor of cell killing CO₃^{·-} and/or ^{·}OH is consumed in, on, or near the implant, or the transplanted tissue, organ, or cell is reduced, without substantially affecting the concentration of OONO⁻, or O₂^{·-}, in tissues or organs remote from the implant or transplant. Preferably, the catalyst affects the concentration of OONO⁻, or O₂^{·-} locally, not systemically. The preferred catalysts do not affect the concentrations of OONO⁻ or O₂^{·-} in organs or tissues at a distance greater than about 5 cm from the implant or transplant, preferably do not affect these at a distance greater than about 2 cm from the implant or transplant, and most preferably they do not affect these at a distance greater than about 1 cm from the implant or transplant.

The model of the amplified cell killing cycle, disrupted by the immobilized catalysts of this invention, by which this invention is not being limited, is the following. The CO₃^{·-} -radical formed, for example, by Reaction 2 or by Reaction 3, and the ^{·}OH radicals, formed by decomposition of the peroxynitrite anion, are cell killing oxidants. When a cell dies naturally, by the orchestrated process of programmed cell death, its decomposition products are not chemo-attractants of macrophages or other killer cells. In contrast, when a cell is killed by a product of a reaction of ONOO⁻, molecules released by, or debris produced of, the dead cells is chemotactic for (chemically attracts, or “recruits” more) killer cells and/or their progenitors, such as monocytes, macrophages and/or neutrophils. The greater the number of the cells killed, the greater the number of killer cells or killer cell progenitors recruited by the chemotactic molecules released from, and/or chemotactic debris from, the dead cells. The greater the number of, or the coverage of the transplant by, debris-recruited macrophages, the greater the rate of local generation of the two precursors of which the peroxynitrite killer anions are spontaneously formed, which are nitric oxide (^{·}NO) and the superoxide radical anion (O₂^{·-}). The result is a cell death-amplified, peroxynitrite anion-mediated, feedback loop, resulting in a flare up in which more of the transplanted cells are killed. This self propagating, progressively more destructive cycle can be slowed or prevented by reducing the local concentration of peroxynitrite anions through an immobilized catalyst accelerating their isomerization, or accelerating the decay of their O₂^{·-} precursor.

The catalyst can be immobilized on the implant prior to implantation. Optionally, it can be slowly released after implantation. Alternatively, it can be in a hydrogel immobilized on the surface of the implant. The preferred hydrogels are permeable to ONOO⁻ and/or to NO₃⁻ and/or to O₂ and/or H₂O₂. The catalyst can be incorporated in, on, or near a transplant after transplantation, or it can be incorporated in or on the transplant after its removal from the donor but prior to transplantation in the recipient. The catalyst can be a polymer-bound molecule or ion, bound within the polymer by electrostatically, and/or coordinatively and/or covalently and/or through hydrogen bonding, and/or through hydrophobic interaction. The preferred polymers, to which the catalyst is bound, swell, when immersed in a pH 7.2 solution containing 0.14 M NaCl at 37° C. to a hydrogel.

The immobilized, or slowly leached, catalyst can lower near the implant, or near the transplant, or near an inflamed organ, such as the skin after it is burned, the local concentration of OONO⁻ through its isomerization reaction OONO⁻→NO₃⁻, or through any reaction of its precursor O₂^{·-}, other than combination with ^{·}NO, whereby ONOO⁻ would be formed. Preferably, the catalyst lowering the O₂^{·-} concentration contains osmium and most preferably it dismutates O₂^{·-} through Reaction 4, O₂^{·-}+2H⁺→H₂O₂+O₂. The preferred ONOO⁻ isomerization catalysts are natural or man-made macromolecules comprising a transition metal complex of a macrocycle, such as an iron porphyrin or a manganese porphyrin. (See, for example, “Mn(II)-Texaphyrin as a Catalyst for the Decomposition of Peroxynitrite”. R. Shimanovich et al., Journal of the American Chemical Society (2001), 123(15), 3613-3614; Reaction of Human Hemoglobin with Peroxynitrite: Isomerization to Nitrate and Secondary Formation of Protein Radicals. N. Romero et al., Journal of Biological Chemistry (2003), 278(45), 44049-44057. The catalyst can also be an enzyme, such as one of the enzymes of Herold et al. “Mechanistic Studies of the Isomerization of Peroxynitrite to Nitrate Catalyzed by Distal Histidine Metmyoglobin Mutants”, Journal of the American Chemical Society, Web publication date May 12, 2004. According to Herold et al., the iron(III) forms of the sperm whale myoglobin mutants H64A, 1464D, H64L, F43W/H64L, and H64Y/H93G catalyze efficiently the isomerization of peroxynitrite to nitrate.

Peroxynitrite isomerization catalysts. Peroxynitrite anion, ONOO⁻, isomerization catalysts, catalyzing the reaction ONOO⁻→NO₃⁻, can be applied, according to this invention, in hydrogels on implants or in hydrogels in, on or near transplants. The hydrogels comprise a preferably crosslinked polymer, such as a co-polymer of acrylamide, swelling at about 37° C. in a pH 7.2-7.4 phosphate buffer solution, containing 0.14 M NaCl, to a hydrogel containing at least 20 weight % water, preferably at least 40 weight % water and most preferably at least 60 weight % water. The hydrogels are permeable to ONOO⁻ or to NO₃⁻. The useful hydrogels of this invention can contain either protein-based or non-protein based isomerase. Examples of protein based isomerases are provided in the study of S. Herold et al. “Mechanistic Studies of the Isomerization of Peroxynitrite to Nitrate Catalyzed by Distal Histidine Metmyoglobin Mutants”, Journal of the American Chemical Society, Web publication date May 12, 2004. Herold et al. found that the iron(III) forms of the sperm whale myoglobin mutants H64A, H64D, H64L, F43W/H64L and H64Y/H93G efficiently catalyze the isomerization of peroxynitrite to nitrate. Appropriate hydrogels and methods of binding enzymes within hydrogels are well known. See, for example, “Long tethers binding redox centers to polymer backbones enhance electron transport in enzyme” Wiring “hydrogels” F. Mao, N. Mano and A. Heller Journal of the American Chemical Society, 125(16), 4951-7 (2003). Isomerization catalysts, which unlike those of Herold do not contain proteins, were also described in patents and research articles. The catalysts are usually metal, mostly manganese or iron, complexes of macrocycles, like phthalocyanines or porphyrins. Citing M. P. Jensen and D. P. Riley, “Peroxynitrite is decomposed catalytically by micromolar concentrations of water-soluble Fe(III) porphyrin complexes, including 5,10,15,20-tetrakis(2′,4′,6′-trimethyl-3,5 disulfonatophenyl) porphyrinato ferrate (7-), Fe(TMPS); 5,10,15,20-tetrakis(4′-sulfonatophenyl) porphyrinatoferrate(3-), Fe(TPPS); and 5,10,15,20-tetrakis(N-methyl-4′-pyridyl)porphyrinatoiron(5+), Fe(TMPyP). Spectroscopic (UV-visible), kinetic (stopped-flow), and product (ion chromatographic) studies reveal that the catalyzed reaction is a net isomerization of peroxynitrite to nitrate (NO3-). One-electron catalyst oxidation forms an oxoFe (IV) intermediate and nitrogen dioxide, and recombination of these species is proposed to regenerate peroxynitrite or to yield nitrate. (“Peroxynitrite Decomposition Activity of Iron Porphyrin Complexes” Inorganic Chemistry 2002, 41, 4788-4797). According to R. Shimanovich and co-workers Mn (II)-texaphyrin catalyzes the decomposition of peroxynitrite. (“Mn (II)-Texaphyrin as a Catalyst for the Decomposition of Peroxynitrite” Journal of the American Chemical Society, 2001, 123, 3613-3614). J. Lee et al., “Mechanisms of Iron Porphyrin Reactions with Peroxynitrite.”, Journal of the American Chemical Society, 1998, 120, 7493-7501 state that “water-soluble iron porphyrins, such as 5,10,15,20-tetrakis(N-methyl-4′-pyridyl)porphinatoiron(III) [Fe(III)TMPyP] and 5,10,15,20-tetrakis(2,4,6-trimethyl-3,5-sulfonatophenyl) porphinatoiron(III) [Fe(III)TMPS] catalyze the efficient decomposition of ONOO⁻ to NO₃⁻ and NO₂⁻ under physiological conditions. Hemoglobin also catalyzes the isomerization reaction. (“Reaction of Human Hemoglobin with Peroxynitrite: Isomerization to Nitrate and Secondary Formation of Protein Radicals” N. Romero et al., Journal of Biological Chemistry (2003), 278(45), 44049-44057) According to this invention, the complexes, such as those described by Jensen and Riley, would be slightly modified by well established procedures to add a linkable function, such as carboxylate, or amine, then covalently bound by forming amides with amine, or carboxylate functions of the polymer of the hydrogel. See, for example, “Long tethers binding redox centers to polymer backbones enhance electron transport in enzyme “Wiring” hydrogels” F. Mao, N. Mano and A. Heller Journal of the American Chemical Society, 125(16), 4951-7 (2003).

Acceptable and unacceptable surface topographies of implants. According to the model applied in this invention, the immune system and its killer cells evolved to fight invading pathogens, not implants or transplants, which were only recently introduced in the human body. Hence, it is best adapted to recognize and kill pathogens, particularly the most frequently invading pathogens, which are bacteria. The killer cells phagocytize (engulf in phagosomes) the invaders. According to this invention, killer cells, like macrophages, are recruited by, adhere to and merge on, implants, exemplified by stents, if they have surface features, particularly protruding features of dimensions similar to those of bacteria, which are misinterpreted by the immune system as pathogens. Such features must be avoided.

Macrophages and/or neutrophils, which are phagocytes, engulf and seal bacteria, as well as other particles having dimensions similar to those of bacteria, in phagosomes. As the phagocytes, which are killer cells of this invention, are recruited, and their density on the surface increases, the local concentrations of the two phagocyte/killer cell generated pre-precursors, O₂^{·-} and ·NO, increases and with it the concentration of the ONOO⁻ precursor, of the two cell-killing CO₃^{·-} and ^{·}OH radicals. Upon their killing of healthy tissue cells near the implant chemotactic molecules and/or debris is released from the killed cells, and more killer cells are recruited, their secretion of O₂^{·-} and ^{·}NO further raising the concentration of ONOO⁻ and the cell killing radicals, resulting in an amplified cycle leading to massive killing of cells and the formation of a lesion near the implant. Its repair, by fibrotic tissue, underlies the proliferation of cells near stents and other implants.

Pathogenic microorganisms in humans, which phagocytes could engulf, range in their dimensions from about 0.1 μm to about 100 μm, the respective dimensions of viruses and amoebae. The most common and the most relevant of these are, in the context of implants such as stents, bacteria, many of which adhere to and colonize blood vessel surfaces. Neutrophils, as well as macrophages and giant cells formed of macrophages, are likely to have evolved to phagocytize and kill these. Table 4 shows the dimensions and shapes of 44 bacteria found in humans. The average length of these is 2.61 μm and the average width 0.73 μm, resulting in an average aspect ratio of about 3.6. The shortest bacterium is 0.55 μm long and the longest is 9 μm long; the diameter of the narrowest is 0.1 μm and that of the thickest it is 1.3 μm. Fungal and mycotic disease-causing organisms have diameters of about 5 μm, and dimensions of amoebae reach 100 μm.

TABLE 4
Widths and lengths of 44 human bacteria

| Genus | Species | Strain | Shape | Diameter, μm | Length, μm |
|---|---|---|---|---|---|
| Chlamydia | pneumoniae | AR39 | C | 1 | 1 |
| Chlamydia | pneumoniae | J138 | C | 1 | 1 |
| Escherichia | coli | K12-MG1655 | R | 1.3 | 4 |
| Escherichia | coli | 0157:H7 EDL933 | R | 1.3 | 4 |
| Escherichia | coli | 0157:H7 Sakai | R | 1.3 | 4 |
| Escherichia | coli | UPEC-CFT073 | R | 1.3 | 4 |
| Leptospira | interrogans | str. 56601 | S | 0.1 | 9 |
| Listeria | innocua | Clip11262 | R | 0.45 | 1.5 |
| Listeria | monocytogenes | EGD-e | R | 0.45 | 1.5 |
| Mycobacterium | leprae | TN | R | 0.35 | 4.5 |
| Mycobacterium | tuberculosis | CDC 1551 | R | 0.45 | 2.5 |
| Mycobacterium | tuberculosis | H27Rv | R | 0.45 | 2.5 |
| Mycoplasma | penetrans | HF-2 | FL | 0.3 | 1.4 |
| Neisseria | meningitidis | Z2491 | C | 0.8 | 0.8 |
| Pasteurella | multocida | Pm70 | R | 0.3 | 1.55 |
| Pseudomonas | putida | KT2440 | R | 0.9 | 0.3 |
| Rickettsia | conorii | Malish 7 | R | 0.4 | 1.4 |
| Salmonella | typhi | CT18 | R | 1.1 | 3.5 |
| Salmonella | typhimurium | SGSC1412 | R | 1.1 | 3.5 |
| Staphylococcus | aureus | Mu50 | C | 1 | 1 |
| Staphylococcus | aureus | MW2 | C | 1 | 1 |
| Staphylococcus | aureus | N315 | C | 1 | 1 |
| Streptococcus | agalactiae | NEM316 | C | 0.9 | 0.9 |
| Streptococcus | pneumoniae | R6 | C | 0.875 | 7.8 |
| Streptococcus | pyogenes | SF370(M1) | C | 0.75 | 0.75 |
| Streptococcus | pyogenes | MGA58232 | C | 0.75 | 0.75 |
| Yersinia | pestis | CO92 | C | 0.65 | 2 |
| Yersinia | pestis | KIM5 P12 | C | 0.65 | 2 |
| Mycoplasma | genitalium | G-37 | FL | 0.55 | |
| Ureaplasma | urealyticum | | C | 0.55 | 0.55 |
| Mycoplasma | pneumoniae | M129 | FL | 0.55 | |
| Rickettsia | prowazekii | Madrid E | R | 0.4 | 1.4 |
| Treponema | pallidum | | S | 0.14 | 11.5 |
| Chlamydia | trachomatis | D/UW-3/CX | C | 1 | 1 |
| Chlamydia | pneumoniae | CWL029 | C | 1 | 1 |
| Helicobacter | pylori | J99 | HR | 0.75 | 3 |
| Haemophilus | influenzae | RD | R | 0.4 | 1.75 |
| Helicobacter | pylori | 26695 | HR | 0.75 | 3 |
| Neisseria | meningitidis | MC58 | C | 0.8 | 0.8 |
| Streptococcus | mutans | UA159 | C | 0.625 | 0.625 |
| Campylobacter | jejuni | NCTC11168 | HR | 0.35 | 0.65 |
| Streptococcus | agalactiae | 2603V/R | C | 0.9 | 0.9 |
| Fusobacterium | nucleatum | ATCC 25586 | R | 0.55 | 6.5 |
| Streptococcus | pneumoniae | TIGR4 | C | 0.875 | 7.8 |
| | | Average | | 0.73 | 2.61 |

Shapes:
C—spherical (circular);
R—rod;
S—spiral;
HR—helical rod;
FL—no shape, flexible.

The features likely to be phagocytized on stents and other implants are protrusions having dimensions similar to human pathogens, larger than about 0.1 μm and smaller than about 100 μm. The features that are most likely to be phagocytized have bacterial dimensions. These are typically larger than about 0.2 μm and smaller than about 10 μm. Thus, polishing to remove surface features smaller than about 0.1 μm is costly and has no advantage. Similarly, features greater than about 100 μm should be acceptable. Surface features of dimensions larger than about 0.2 μm and smaller than about 10 μm should be strictly avoided and the most preferred implants and stents should have the least possible surface density of features of such dimensions. It is preferred that features of dimensions larger than about 0.1 μm and smaller than about 100 μm also be avoided. Features smaller than about 0.1 μm or larger than about 100 μm are acceptable.

In general, it is desired that there be as few as possible, or preferably no features that are phagocytized on the surface of the implant or, when the implant is coated, on its coating. The stents or other implants are increasingly more preferred when the number of phagocytized features per square millimeter decreases from about less than about 10³ to less than about 10², to less than about 10¹, to less than about 10⁻¹, to less than about 10⁻², to less than about 10⁻³, to less than about 10⁻⁴. Because phagocytes may have evolved to engulf pathogenic organisms, implant and/or implant coating surfaces, with the fewest features, particularly the fewest protruding surface features of dimensions similar to those of pathogens, are preferred. The fewer of these features, the more the implant and/or its coating are preferred. Thus the implants are increasingly preferred when the number of protruding surface features per square millimeter decreases in from about 10³, to less than about 10², to less than about 10¹, to less than about 10⁻¹, to less than about 10⁻², to less than about 10⁻³, to less than about 10⁻⁴. Adhesion of killer cells or their progenitor cells, such as macrophages or monocytes, to surfaces, is generally indicative of phagocytized featured. In the phagocytized features the pH is lower than the pH in the cytoplasm of the phagocyte. Thus, staining with an indicator changing color at a pH between about 7.35 and about 5.0, preferably between about 6.8 and about 5.5, and most preferably between about 6.5 and about 5.8, would be a useful test for phagocytization of surface features of implants.

The undesired surface features can be removed by electrochemical polishing in the appropriate electrolytic solution and in the appropriate temperature range. Thus, for example the roughness achieved by C. A. Huang et al., Corrosion Science (2003), 45(11), 2627-2638 electropolished high-speed tool steel (ASP 23) using HClO₄—CH₃COOH mixed acids in the temperature range from −10 to 30° C. to obtain an acceptable surface roughness of 30-50 nm.

Preferred metals and alloys for implants. The cell killing radicals CO₃^{·-} and/or ^{·}OH, generated from their precursor ONOO⁻ which is formed of the killer cell generated ^{·}NO and O₂^{·-}. Reactions catalyzed by transition metal ions, such as those of Equations 6-12, may increase the yield, concentration, or rate of formation of cell killing radicals, and may add a path to their formation from H₂O₂, produced in the dismutation reaction of O₂^{·-}. The transition metal ion caused increment in cell killing radicals can be avoided by excluding, or reducing the atom %, of transition metals from the metallic alloys or ceramics used in implants, such as stents. The transition metals to be partly or completely excluded are those that upon their corrosion in physiological buffer solution, serum, plasma or blood release a catalytic transition metal ion.
Mⁿ⁺→M⁽ⁿ⁺¹⁾⁺+e⁻ (6)
e⁻+ONOO⁻+CO₂→CO₃^{·-}+^{·}NO₂⁻ (7)
e⁻+H₂O₂→HCO₃⁻→CO₃^{·-}+H₂O+OH⁻ (8)
e⁻+H₂O₂→^{·}OH+OH⁻ (9)
e⁻+ONOO⁻+H⁺→^{·}OH+^{·}NO₂⁻ (10)
^{·}OH+HCO₃⁻→CO₃^{·-}+H₂O (11)
M⁽ⁿ⁺¹⁾+Cyt_{red}→Mⁿ⁺+Cytₒₓ (12)

Cu⁺, Fe²⁺, Co²⁺ or Ni²⁺ are examples of the reduced transition metal ions Mₙ₊ in Reactions 6 and 12. They are constituents of copper alloys like brass or bronze, stainless steels, cobalt-chromium alloys and nickel-titanium alloys. These ions donate electrons to oxidizers to form the M⁽ⁿ⁺¹⁾ (Reaction 6), such as Cu²⁺, Fe³⁺, Co³⁺ or Ni³⁺. If the ions are reduced by reductants present in the cytoplasm of cells, such as NADH, NADPH, FADH₂, or reduced cytochrome C, Cyt_{red}, (Equation 12) the ions can act as electron sources in reactions such as Reactions 7-10 and catalyze the formation of the cell killing radicals. Indeed, copper-induced inflammatory reaction of rat carotid arteries, mimicking restenosis, has been reported, (see, for example, W. Volker et al., “Copper-induced inflammatory reactions of rat carotid arteries mimic restenosis/arteriosclerosis-like neointima formation” Atherosclerosis, 1997, 130(1-2), 29-36)). Copper induced restenosis was until now unexplained. It is now explained by the teachings of this invention. The preferred implants contain less than 1 atom % of the catalytic transition metal atoms and preferably less than 0.1 atom % of these atoms.

Preferably, the metals, or metallic alloys, or ceramics of implants of this invention contain less than about 1 atom %, and most preferably less than 0.1 atom % of those transition metals that introduce upon their corrosion in physiological buffer solution, and/or in serum, and/or in plasma and/or in blood catalytic transition metal cations. The excluded transition metals increase, by 10% or more, at about 37° C., the yield of CO₃^{·--} and/or ^{·}OH in a pH 7.2-7.4 aqueous solution of either 1 mM ONOO⁻, and/or 1 mM H₂O₂, containing about 10 mM total carbon as HCO₃⁻ and CO₂, and about 0.14 M NaCl.

Acceptable metallic constituent atoms of metallic or ceramic implants, that do not corrode to introduce catalytic transition metal ions, are yttrium, zirconium, hafnium, and magnesium, calcium, aluminum, lithium and scandium. In ceramics, their oxides are preferred. Of these, zirconium is most preferred. For stents, particularly coronary stents, the preferred implant materials are ductile, with a % elongation at failure greater than about 20% at ambient temperature, near 25° C. The % elongation at failure of the most preferred stent alloys is greater than about 30%. Preferred stent and implant alloys include those of the composition ZrₘHfₙ, where m is between about 95 atom %, and 100 atom % and n is between about 0 and about 5 atom %. In the most preferred ZrₘHfₙ alloys m is between 98 atom % and 100 atom %, and n is between about 0 and about 2 atom %. The preferred yttrium, zirconium, hafnium, and scandium alloys and most preferred zirconium alloys contain preferably less than 0.1 atom % of the catalytic transition metals.

Inflammatory reaction to subcutaneously implanted metal wires. Sterilized 0.25 mm wires, purchased from Alfa Asear, Ward Hill, Mass. were implanted subcutaneously in the two arms of the inventor at a depth of about 1 cm. The distance between the implants was about 4-5 cm. After implanting, the external part of the wires was trimmed to about 1 cm and glued to skin, then coated with J&J Liquid Plaster. After 36 h the skin near the copper wire was intensely inflamed. The skin was red across a 3 cm diameter zone surrounding the implant. The skin near the tantalum wire was inflamed; that near the hafnium, tungsten and 304 stainless steel wires was very slightly inflamed, with very small red dots of 1-2 diameters near the wire. The skin near the zirconium wire was not inflamed at all. There was no visible reddening of the skin.

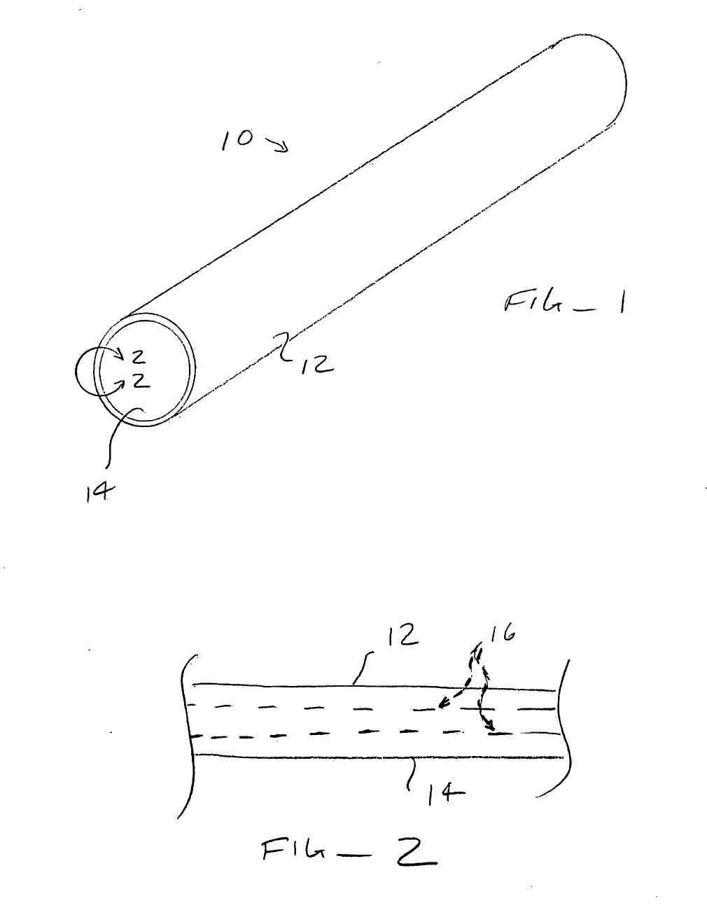

An exemplary implant 10 in the form of a stent or other prosthesis is illustrated in FIG. 1. The medical implant will have an outer or exterior surface 12 which will be exposed to a vascular or tissue environment when implanted in a patient. Optionally, the implant 10 may also have an interior surface 14 which is also exposed to a vascular, tissue, or other environment when implanted.

Thus, in the embodiments of the present invention involving coatings, at least a portion of the exterior surface 12 and/or interior surface 14 will be coated with a hydrogel or other material capable of promoting the isomerization of peroxynitrite anion to nitrate anion. In the second embodiment of the present invention, the surfaces 12 and/or 14 will be fabricated, modified, polished, treated, coated, or otherwise adapted or configured to have a smooth, feature-free surface as described in detail hereinabove. In the third embodiment of the present invention, at least a portion of the metallic body of the implant 10 near surface 12 and/or 14 will be composed of a preferred metal in order to inhibit adverse inflammation. It should be appreciated that the interior portion of the implant 10, as schematically illustrated by broken lines 16 could be composed of any material since they are not exposed to the vascular, tissue, or other patient environment.

While the above is a complete description of the preferred embodiments of the invention, various alternatives, modifications, and equivalents may be used. Therefore, the above description should not be taken as limiting the scope of the invention which is defined by the appended claims.

1. An implant or transplant which has been fabricated or modified to promote the isomerization of peroxynitrite anion to nitrate anion.

2. An implant or transplant as in claim 1, wherein at least a portion of a surface is coated with a catalyst which promotes said isomerization.

3. An implant or transplant as in claim 2, wherein said catalyst is a protein, an enzyme and/or contains a metal complex.

4. An implant as in claim 3, wherein the catalyst is a permeable hydrogel containing a porphyrin and/or phthalocyanine of a transition metal.

5. An implant as in claim 4, wherein the transition metal comprises iron and/or manganese.

6. A method for inhibiting inflammation associated with implantation or transplantation in a patient, said method comprising:
coating at least a portion of an implant device or transplantation structure with a material which catalyzes the isomerization of peroxynitrite anion to nitrate anion.

7. A method as in claim 6, wherein said material comprises a catalyst which promotes said isomerization.

8. A method as in claim 7, wherein said catalyst is a protein, an enzyme and/or contains a metal complex.

9. A method as in claim 8, wherein the catalyst is a permeable hydrogel containing a porphyrin and/or phthalocyanine of a transition metal.

10. A method as in claim 9, wherein the transition metal comprises iron and/or manganese.

11. A hydrogel for coating a medical implant or transplant, said hydrogel comprising a catalyst which promotes the isomerization of peroxynitrite anion to nitrate anion.

12. A hydrogel as in claim 11, wherein said catalyst is a protein, an enzyme and/or contains a metal complex.

13. A hydrogel as in claim 12, wherein the catalyst is a permeable hydrogel containing a porphyrin and/or phthalocyanine of a transition metal.

14. A hydrogel as in claim 13, wherein the transition metal comprises iron and/or manganese.

15. A hydrogel as in claim 14, comprising a co-polymer of acrylamide.

16. A medical implant having an exterior surface, said exterior surface having features with dimensions which are in a size range characteristic of pathogenic bacteria present at a surface density below a threshold value which promotes phagocytosis.

17. An implant as in claim 16, wherein the feature size range is from 0.1 μm to 100 μm.

18. An implant as in claim 17, wherein the threshold surface density is 1000 features per mm².

19. A method for fabricating a medical implant, said method comprising fabricating, treating, or coating at least an exterior surface of the implant so that said surface has features with dimensions which are in a size range characteristic of phagocytosis bacteria present at a surface density below a threshold value which promotes phagocytosis.

20. A method as in claim 19, wherein the feature size range is from 0.1 μm to 100 μm.

21. A method as in claim 20, wherein the threshold surface density is 1000 features per mm².

22. A medical implant having a surface which is substantially free from transition metals which form dissolved ions which catalyze the formation of cell killing radicals.

23. A medical implant as in claim 22, wherein said transition metals are present at or near the surface at an atomic percent below 1%.

24. A medical implant as in claim 23, wherein said transition metals include cooper, iron, cobalt, and nickel.

25. A medical implant as in claim 24, wherein said surface is at least partly composed of a metal selected from the group consisting of yttrium, zirconium, hafnium, magnesium, calcium, aluminum, lithium, and scandium or any of their alloys, or their oxides.

26. A medical implant as in any of claims 22 to 25, wherein the implant is composed of a metal or alloy having a 20% or great elongation failure at room temperature.

27. A medical implant as in claim 22, wherein the implant is a stent composed of at least 95 atomic percent zirconium with from 0 to 5 atomic percent hafnium.

28. A method for fabricating a medical implant, said method comprising forming at least a surface portion of the implant from a material which is substantially free from transition metals which form dissolved ions which catalyze the formation of cell killing radicals.

29. A method as in claim 28, wherein said transition metals are present at or near the surface at an atomic percent below 1%.

30. A method as in claim 29, wherein said transition metals include cooper, iron, cobalt, and nickel.

31. A method as in claim 30, wherein said surface is at least partly composed of a metal selected from the group consisting of yttrium, zirconium, hafnium, magnesium, calcium, aluminum, lithium, and scandium or any of their alloys, or their oxides.

32. A method as in any of claims 28 to 31, wherein the implant is composed of a metal or alloy having a 20% or great elongation failure at room temperature.

33. A method as in claim 28, wherein the implant is a stent composed of at least 95 atomic percent zirconium with from 0 to 5 atomic percent hafnium.
